(19) United States
(12) Patent Application Publication
(10) Pub. No.: US 2023/0285049 A1
(43) Pub. Date: Sep. 14, 2023

(54) Intraosseous Access System

(71) Applicant: Bard Access Systems, Inc., Salt Lake City, UT (US)

(72) Inventor: Glade H. Howell, Draper, UT (US)

(21) Appl. No.: 18/120,863

(22) Filed: Mar. 13, 2023

Related U.S. Application Data
(60) Provisional application No. 63/319,671, filed on Mar. 14, 2022.

(51) Int. Cl. A61B 17/34

(52) U.S. Cl. CPC A61B 17/3472; A61B 2017/00526

(57) ABSTRACT

An intraosseous access system including an intraosseous driver coupleable with access assembly that includes an obturator assembly having an obturator and an access assembly needle assembly having a needle lumen defining a needle lumen. The access assembly further includes an extension set having an extension tube having extension tube hub and a bag attachment member coupled with the extension tube at opposite ends. The extension tube hub forms a rotating seal with the access assembly. The access assembly and the bag attachment member are contained within separate sterile packages with the extension tube extending therebetween. A fluid pathway extends between the bag attachment member and the needle lumen.

### PRIORITY

This application claims the benefit of priority to U.S. Provisional Application No. 63/319,671, filed Mar. 14, 2022, which is incorporated by reference in its entirety into this application.

### BACKGROUND

Intraosseous access systems may be used to quickly access the bone marrow cavity. In many configuration, intraosseous drivers rotate to drill an intraosseous needle assembly having an obturator and an intraosseous needle into the bone marrow to access the bone marrow cavity. Once the intraosseous needle assembly accesses the bone marrow cavity, the obturator is removed, exposing the intraosseous needle to potential contamination while an extension tube is coupled to the intraosseous needle. It would be beneficial to the patient and the user to have an intraosseous access system that has the extension tube pre-connected, reducing a connection step and the risk of contamination. Disclosed herein is an intraosseous access system and method of use that addresses the foregoing.

### SUMMARY

Disclosed herein is an intraosseous access system that, according to some embodiments, includes an intraosseous driver and an access assembly coupled with the driver, where the access assembly includes obturator assembly including an obturator and a needle assembly including an access needle, and where the needle assembly is coupled with the obturator assembly such that the obturator is disposed within a needle lumen of the access needle. The intraosseous access system further includes an extension set coupled with the access assembly, where the extension set includes an extension tube extending between an extension tube hub at a proximal end and a bag attachment member at a distal end, and where the extension tube hub is coupled with the access assembly such that bag attachment member is in fluid communication with the needle lumen.

In some embodiments, the access assembly is rotatably coupled with the extension tube hub to enable the access assembly to rotate with respect to the extension set.

In some embodiments, the needle assembly includes a fluid port in fluid communication with the needle lumen, and the extension tube hub includes (i) a first O-ring defining a first rotating seal with the needle assembly, where the first O-ring is disposed proximal the fluid port; and (ii) a second O-ring defining a second rotating seal with the needle assembly, where the second O-ring is disposed distal the fluid port.

In some embodiments, during use, the bag attachment member is coupled with a fluid bag having a fluid therein, and in some embodiments, during use, the fluid flows from the fluid bag through needle lumen.

In some embodiments, the system further includes a first sterile package, where the access assembly is contained within the first sterile package. In some embodiments, the extension tube extends between a sterile interior of the first sterile package and a non-sterile exterior of the first sterile package.

In some embodiments, the system further includes a second sterile package, where the bag attachment member is contained within the second sterile package and in some embodiments, the extension tube extends between a sterile interior of the second sterile package and a non-sterile exterior of the second sterile package.

Also disclosed herein is a method of accessing an intraosseous cavity that, according to some embodiments, includes (i) coupling an intraosseous driver to an access assembly of an intraosseous access system; (ii) activating the intraosseous driver to access the intraosseous cavity, where activating the intraosseous driver includes rotating an access needle the access assembly; (iii) coupling an extension set of the access assembly to a fluid bag containing a fluid, where the extension set is rotatably coupled with the access assembly to enable rotation of the access assembly with respect to the extension set; and (iv) delivering the fluid to the intraosseous cavity through the extension set and a needle lumen of the needle.

In some embodiments of the method, activating the intraosseous driver and delivering the fluid occur simultaneously.

In some embodiments of the method, the intraosseous access system includes a first sterile package containing the access assembly, where at least a first portion of an extension tube of the extension set is contained within the first sterile package, and where at least a second portion of the extension tube is not contained within the first sterile package. In such embodiments, the method further includes removing the access assembly from the first sterile package. In some embodiments of the method, the extension tube extends between a sterile interior of the first sterile package and a non-sterile exterior of the first sterile package, and the first sterile package forms a seal with the extension tube.

In some embodiments of the method, the intraosseous access system includes a second sterile package containing a bag attachment member of the extension set, where at least a third portion of the extension tube is contained within the second sterile package, and where at least a fourth portion of the extension tube is not contained within the second sterile package. In such embodiments, the method further includes removing the bag attachment member from the second sterile package. In some embodiments of the method, the extension tube extends between a sterile interior of the second sterile package and a non-sterile exterior of the second sterile package, and the second sterile package forms a seal with the extension tube.

Also disclosed herein is a method of manufacturing an intraosseous access system that, according to some embodiments, includes (i) forming an extension set that includes an extension tube having an extension tube hub at a first end and a bag attachment member at a second end; and (ii) coupling the extension tube hub with an access assembly, where the access assembly includes an obturator assembly having an obturator and a needle assembly having an access needle, where the obturator assembly is coupled with the needle assembly such that the obturator is disposed within a needle lumen of the access needle. In accordance with such embodiments, coupling the extension tube hub with the access assembly includes (i) encapsulating the extension tube hub between the obturator assembly and the needle assembly and (ii) forming a rotating fluid-tight seal between the extension tube hub and the access assembly.

In some embodiments of the manufacturing method, coupling the extension tube hub with the access assembly further includes defining a fluid pathway between the bag attachment member and the needle lumen.

In some embodiments, the manufacturing method further includes containing the access assembly within a first sterile package such that at least a first portion of the extension tube is contained within the first sterile package and at least a second portion of the extension tube is not contained within the first sterile package.

In some embodiments, the manufacturing method further includes containing the bag attachment member within a second sterile package, separate from the first sterile package, such that at least a third portion of the extension tube is contained within the second sterile package and at least a fourth portion of the extension tube is not contained within the second sterile package.

In some embodiments of the manufacturing method containing the access assembly within the first sterile package includes forming a seal between the first sterile package and the extension tube. In some embodiments of the manufacturing method containing the bag attachment member within the second sterile package includes forming a seal between the second sterile package and the extension tube.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exploded view of an exemplary intraosseous access system, with some components thereof shown in elevation and another shown in perspective, in accordance with some embodiments;

FIG. 2A illustrates a cross-sectional view of the access assembly including an extension set, in accordance with some embodiments;

FIG. 2B illustrates a cross-sectional view of the access assembly including the extension set within a first sterile package and a bag attachment member within a second sterile package, in accordance with some embodiments;

FIGS. 3A-3C illustrates a cross-sectional view of an exemplary method of accessing an intraosseous cavity and flowing fluid into the intraosseous cavity using the intraosseous access system of FIG. 1 including the extension set of FIG. 2A, in accordance with some embodiments;

FIG. 4 illustrates a flow chart of an exemplary method of accessing an intraosseous cavity using the intraosseous access system, in accordance with some embodiments; and

FIG. 5 illustrates a flow chart of an exemplary method of manufacturing the intraosseous access system, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, an intraosseous access system disclosed herein includes a portion of the intraosseous access system intended to be near a clinician when the intraosseous access system is used on a patient. For example, a “proximal length” of, for example, the intraosseous access needle includes a length of the intraosseous access needle intended to be near the clinician when the intraosseous access needle is used on the patient. A “proximal end” of, for example, the intraosseous access needle includes an end of the intraosseous access needle intended to be near the clinician when the intraosseous access needle is used on the patient. The proximal portion, the proximal-end portion, or the proximal length of the intraosseous access needle can include the proximal end of the intraosseous access needle; however, the proximal portion, the proximal-end portion, or the proximal length of the intraosseous access needle need not include the proximal end of the intraosseous access needle. That is, unless context suggests otherwise, the proximal portion, the proximal-end portion, or the proximal length of the intraosseous access needle is not a terminal portion or terminal length of the intraosseous access needle.

With respect to “distal,” a “distal portion” or a “distal-end portion” of, for example, an intraosseous access system disclosed herein includes a portion of the intraosseous access system intended to be near or in a patient when the intraosseous access system is used on the patient. For example, a “distal length” of, for example, the intraosseous access needle includes a length of the intraosseous access needle intended to be near or in the patient when the intraosseous access needle is used on the patient. A “distal end” of, for example, the intraosseous access needle includes an end of the intraosseous access needle intended to be near or in the patient when the intraosseous access needle is used on the patient. The distal portion, the distal-end portion, or the distal length of the intraosseous access needle can include the distal end of the intraosseous access needle; however, the distal portion, the distal-end portion, or the distal length of the intraosseous access needle need not include the distal end of the intraosseous access needle. That is, unless context suggests otherwise, the distal portion, the distal-end portion, or the distal length of the intraosseous access needle is not a terminal portion or terminal length of the intraosseous access needle.

The phrases “connected to,” “coupled to/with,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

Unless defined otherwise, all technical and scientific terms used herein have the same meaning as commonly understood by those of ordinary skill in the art.

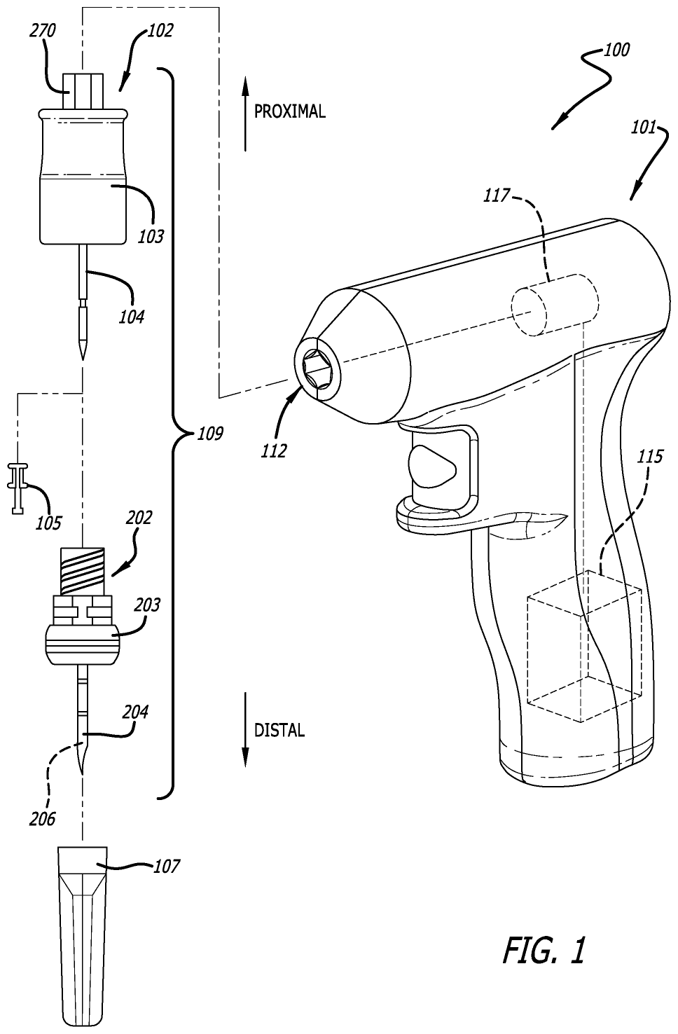

FIG. 1 shows an exploded view of an exemplary intraosseous access system (“system”) 100, with some components thereof shown in elevation and another shown in perspective. In an embodiment, the intraosseous access system 100 can be used to penetrate skin surface tissue layers and underlying hard bone, i.e. bone cortex, for intraosseous access, such as, for example to access the marrow of the bone and/or a vasculature of the patient via a pathway through an interior of the bone, i.e. the medullary cavity. As used herein, an “access event” includes accessing the medullary cavity with an intraosseous access system 100.

In an embodiment, the system 100 includes an intraosseous driver (“driver”) 101 and an access assembly 109. The driver 101 can be used to rotate the access assembly 109 so as to “drill” an access needle 204 into the bone of a patient. In some embodiments, the driver 101 can be automated or manual. As shown in FIG. 1, the driver 101 is an automated driver. For example, the automated driver 101 can be a drill that achieves high rotational speeds. In an embodiment, the access assembly 109 may include an obturator assembly 102 and a needle assembly 202. The needle assembly 202 can include an access needle 204 supported by a needle hub 203. In an embodiment, the obturator assembly 102 includes an elongate obturator 104. As used herein, an obturator 104 includes an elongate medical device configured to be disposed within a needle lumen 206 of the access needle 204 and to prevent bone fragments, tissue, or the like from entering the needle lumen 206. Advantageously, the obturator 104 prevents tissues from obstructing a fluid flow through the needle lumen 206, during or after placement of the access needle 204 to access the medullary cavity with or without the obturator 104 disposed within the needle lumen 206. As will be appreciated, in some embodiments, the obturator 104 may be replaced with a different elongated medical instrument. As used herein, the term “elongated medical instrument” is a broad term used in its ordinary sense that includes, for example, such devices as needles, cannulas, trocars, obturators, stylets, and the like. Accordingly, an obturator assembly 102 may be referred to more generally as an elongated medical instrument assembly. In like manner, the obturator 104 may be referred to more generally as an elongated medical instrument.

In some embodiments, the system 100 may include a safety shield 105 configured to cover the obturator 104 when the obturator 104 is removed from the needle assembly 202. In an embodiment, the shield 105 is configured to couple with the obturator 104 to prevent accidental needle stick injuries when the obturator 104 is removed after placement of the access needle 204.

In an embodiment, the obturator assembly 102 includes an obturator hub 103 coupled with the obturator 104 in any suitable manner (e.g., one or more adhesives or over-molding). The obturator hub 103 can be configured to interface with the driver 101. The obturator hub 103 may alternatively be referred to as an obturator hub or, more generally, as an elongated instrument hub.

In the illustrated embodiment, the needle assembly 202 includes the access needle 204. However, in some embodiments, the access needle 204 may be replaced with a different instrument, such as, for example, a cannula, a tube, or a sheath, and/or may be referred to by a different name, such as one or more of the foregoing examples. Accordingly, the needle assembly 202 may be referred to more generally as a cannula assembly or as a tube assembly. In like manner, the access needle 204 may be referred to more generally as a cannula. In an embodiment, the needle assembly 202 includes a needle hub 203 that is attached to the access needle 204 in any suitable manner. The needle hub 203 can be configured to couple with the obturator hub 103 and may thereby be coupled with the driver 101. The needle hub 203 may alternatively be referred to as a cannula hub. In an embodiment, a cap 107 may be provided to cover at least a distal portion of the access needle 204 and the obturator 104 disposed within the needle lumen 206 prior to use of the access assembly 109. For example, in an embodiment, a proximal end of the cap 107 can be coupled to the obturator hub 103.

In some embodiments, the intraosseous driver 101 can include an energy source 115. In some embodiments, the energy source 115 is configured to energize the rotational movement of a coupling interface 112 and provide a motive force. In some embodiments, the energy source 115 may include one or more batteries that provide electrical power to the driver 101. The energy source 115 may be coupled with the coupling interface 112 via electrical coupling including in some embodiments, an electrical motor that generates rotational movement from electrical energy provided by an electrical energy source 115. The driver 101 can further include a gear assembly 117 configured to translate rotational movement of the electrical motor to rotational movement of the coupling interface 112 and the access assembly 109 coupled thereto. In some embodiments, the obturator assembly 102 may include an intraosseous socket head 270 configured to be detachably coupled to the driver 101.

Further details and embodiments of the intraosseous access system 100 can be found in WO 2018/075694, WO 2018/165334, WO 2018/165339, and U.S. Pat. No. 10,893,887, each of which is incorporated by reference in its entirety into this application.

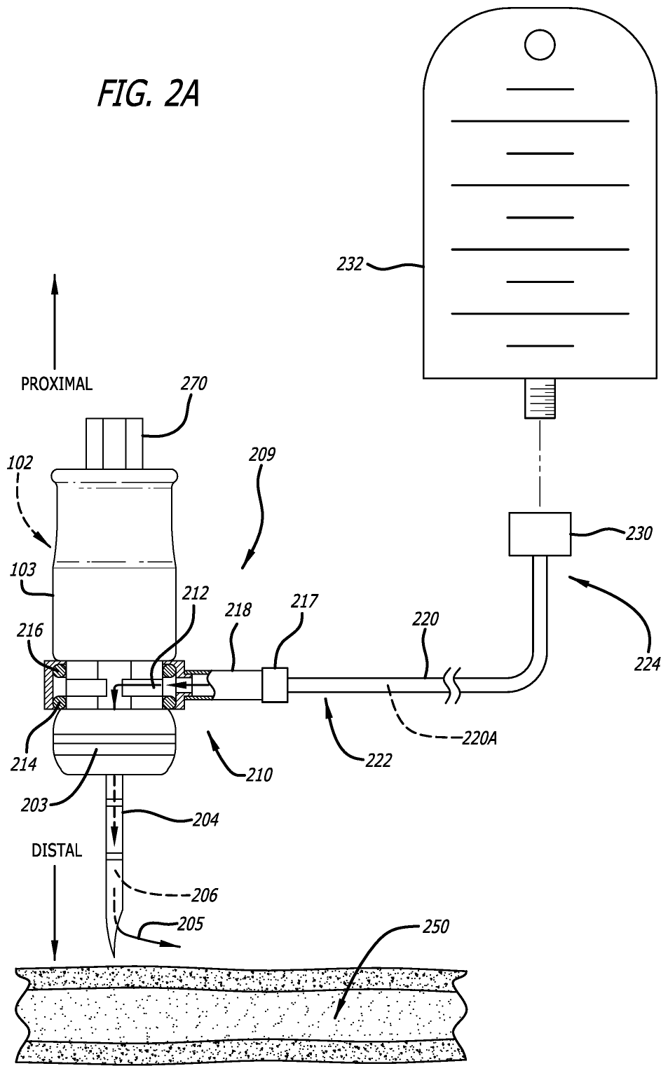

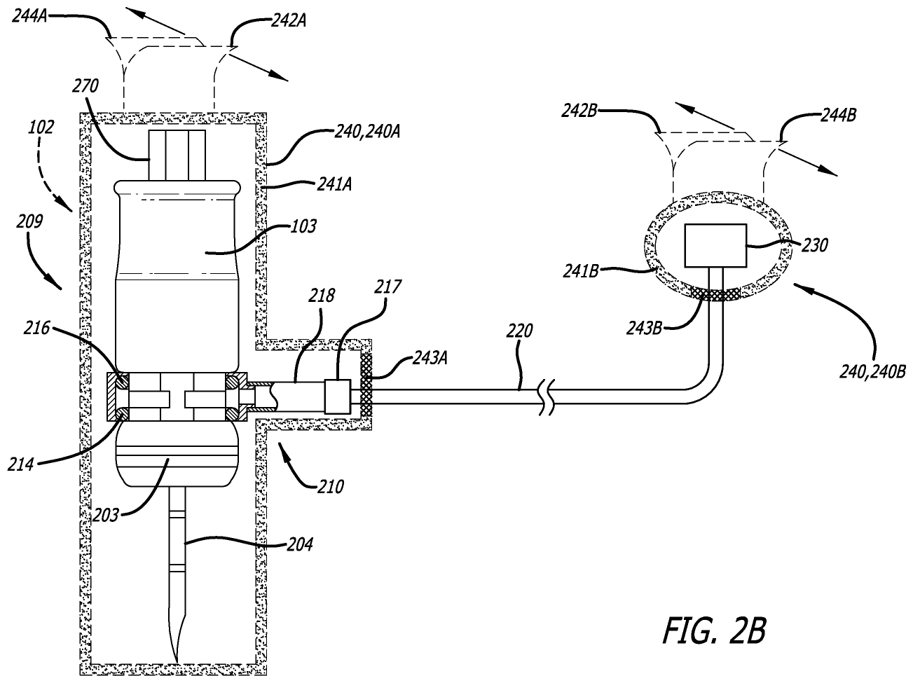

FIG. 2A illustrates a cross-sectional view of an access assembly 209 that may in some respects resemble the features, components, and functionality of access assembly 109 of FIG. 1. The access assembly 209 includes the obturator assembly 102 and the needle assembly 202. The access assembly 209 further includes an extension set 210, in accordance with some embodiments. In some embodiments, the extension set 210 is coupled (e.g., pre-connected) with the access assembly 209. In some embodiments, the access assembly 209 and/or the extension set 210 may be disposable, i.e., configured for single use. In some embodiments, the extension set 210 may be configured for attachment to and detachment from the access assembly 209. The extension set 210 generally defines a fluid pathway 205 between an extension tube lumen 220A of an extension tube 220 and the needle lumen 206, while allowing the driver 101 to rotate the access assembly 209 to access the intraosseous cavity 250. In some embodiments, the extension set 210 may be configured to reduce a connection step between the extension tube 220 and the needle lumen 206 typically performed after the intraosseous cavity 250 is accessed, thereby reducing possible contamination at the connection point of the needle lumen 206 and the extension tube 220 and also reducing the time needed to flow fluids into the intraosseous cavity 250.

In some embodiments, the extension set 210 may include an extension tube hub 218 configured to be coupled between the extension tube and the access assembly 209. In some embodiments, the extension tube hub 218 may extend laterally from the needle assembly 202. The extension tube hub 218 is in fluid communication with the needle lumen 206. In some embodiments, the extension tube hub 218 may be configured to receive thereon or therein the extension tube 220 to facilitate coupling of the extension tube 220 with the extension tube hub 218. The extension tube 220 defines a first end 222 and a second end 224. In some embodiments, the first end 222 may be a distal end and may include an extension tube attachment 217 configured to couple with the extension tube hub 218. In some embodiments, the extension tube attachment 217 may be configured to couple with the extension tube hub 218 via a snap fit, a press fit, an interference fit, a screw fit, or the like. In some embodiments, extension tube attachment 217 may enable detachment of the extension tube 220 from the extension tube hub 218.

The second end 224 may be a proximal end and may include a bag attachment member 230 configured to couple with a fluid bag 232. In some embodiments, the second end 224 may be coupled to the fluid bag 232 in a press fit, a snap fit, a screw fit, an interference fit, or the like. In some embodiments, the bag attachment member 230 may include a Luer lock. In some embodiments, the bag attachment member 230 may enable decoupling of the extension tube 220 from the fluid bag 232. In some embodiments, the extension set 210 may be a separate structure from the access assembly 209 and coupled to the access assembly 209. In other embodiments, the extension set 210 may be integrated into the access assembly 209, i.e., the access assembly 209 may include the extension set 210.

In some embodiments, the access assembly 209 may extend through the extension tube hub 218 and sealably couple with the access assembly 209. In some embodiments, coupling the extension tube hub 218 with the access assembly 209 may include encapsulating the extension tube hub 218 between the obturator assembly 102 and the needle assembly 202. In some embodiments, the extension tube hub 218 may include a proximal seal 216 and a distal seal 214 where each define a rotating fluid-tight seal with the needle hub 203. In some embodiments, the proximal seal 216 may include a first O-ring 216 and the distal seal 214 may include a second O-ring 214. In some embodiments, the first O-ring 216 and the second O-ring 214 may be disposed on proximal side and a distal side, respectively, of a port 212 in fluid communication with the needle lumen 206. Each of the first O-ring 216 and the second O-ring 214 may be configured to provide a fluid-tight seal around needle hub 203. By way of summary, extension tube hub 218 defines a fluid pathway 205 between the fluid bag 232 and the needle lumen 206.

Each of the first O-ring 216 and the second O-ring 214 are configured to allow the access assembly 209 to rotate around a fixed axis independently of (i.e., with respect to) the extension set 210 including the extension tube hub 218 and the extension tube 220 while maintaining the fluid-tight seal. In some embodiments, the first O-ring 216 and the second O-ring 214 may be constructed of rubber, one or more elastomers, or the like. In some embodiments, the first O-ring 216 and second O-ring 214 may be injection molded, stamped from a sheet of material, 3D printed, or the like. In some embodiments, the extension tube hub 218 and the port 212 may be constructed of one or more polymers, one or more alloys, or a combination thereof. In some embodiments, the extension tube hub 218 and the port 212 may be injection molded, 3D printed, extruded, or the like.

FIG. 2B illustrates a cross-sectional view of the obturator assembly 102 coupled with the access assembly 209 including the extension set 210 in sterile package 240, in accordance with some embodiments. In some embodiments, the access assembly 209 including the extension set 210 (or portion thereof) may be secured within the sterile package 240. In some embodiments, the sterile package 240 may be configured for single use. In some embodiments, the sterile package 240 may include more than one sterile package. For example, in the illustrated embodiment, the sterile package 240 includes a first sterile package 240A and a second sterile package 240B where the second sterile package 240B is separate from the first sterile package 240A. In the illustrated embodiment, the access assembly 209 and the extension tube hub 218 may be contained within the first sterile package 240A and the bag attachment member 230 may be contained within the second sterile package 240B. In some embodiments, at least a portion of the extension tube 220 may be disposed outside the sterile package 240. In some instances of use, the user may open the first sterile package 240A and leave the second sterile package 240B closed.

The first sterile package 240A includes a closure mechanism composed of opposing tabs 242A/244A. The opposing tabs 242A/244A are coupled together to form a seal (or at least a sterile barrier) between the opposing tabs 242A/244A. The opposing tabs 242A/244A are configured to be separated (e.g., peeled apart), so as to enable opening of the first sterile package 240A by pulling the opposing tabs 242A/244A apart. In some embodiments, the first sterile package 240A includes opposing panels coupled together via a separable perimeter seam 241A, where the separable perimeter seam 241A is configured for separation (e.g., peeled apart) by pulling apart the opposing panels, e.g., further pulling apart the opposing tabs 242A/244A. As such, during use, the user may open the first sterile package 240A by pulling opposing tabs 242A/244A apart, and the user may continue pull the opposing tabs 242A/244A apart from each other to separate the separable perimeter seam 241A so that the access assembly 209 may be removed from the first sterile package 240A. In some embodiments, the first sterile package 240A may include one or more frangible bands or segments (e.g., perforations) in leu of or in addition to the separable perimeter seam 241A to enable separation of the opposing panels or otherwise facilitate opening of the first sterile package 240A.

In some embodiments, the extension tube 220 may extend from a sterile interior to a non-sterile exterior of the first sterile package 240A. In such embodiments, the first sterile package 240A may form a seal 243A (or at least a sterile barrier) with the extension tube 220 or the extension tube hub 218. In the illustrated embodiment, the first sterile package 240A may be configured for separation from the extension tube 220. In some embodiments, the separable perimeter seam 241A may extend around the extension tube 220 or the extension tube hub 218 to form the seal 243A. As such, pulling the opposing tabs 242A/244A may separate the separable perimeter seam 241A from the extension tube 220. The first sterile package 240A may be formed of any suitable medical grade plastic sheet material and the separable perimeter seam 241A may be formed by a heat sealing process or any other suitable bonding process. Similarly, the seal 243A may also be formed by the heat sealing process or any other suitable bonding process.

Similar to the first sterile package 240A, the second sterile package 240B includes a closure mechanism composed of opposing tabs 242B/244B. The opposing tabs 242B/244B are coupled together to form a seal (or at least a sterile barrier) between the opposing tabs 242B/244B. The opposing tabs 242B/244B are configured to be separated, so as to open the second sterile package 240B by pulling the opposing tabs 242B/244B apart. In some embodiments, the second sterile package 240B includes opposing panels coupled together via a separable perimeter seam 241B, where the separable perimeter seam 241B is configured for separation by pulling apart the opposing panels. As such, during use, the user may open the second sterile package 240B by pulling opposing tabs 242B/244B apart, and the user may continue to pull the opposing tabs 242B/244B apart from each other to separate the separable perimeter seam 241B so that the bag attachment member 230 may be removed from the second sterile package 240B. In some embodiments, the second sterile package 240B may include one or more frangible bands or segments (e.g., perforations) in leu of or in addition to the separable perimeter seam 241B to enable separation of the opposing panels or otherwise facilitate opening of the second sterile package 240B. As the extension tube 220 extends from a sterile interior to a non-sterile exterior of the second sterile package 240B, the second sterile package 240B may form a seal 243B (or at least a sterile barrier) with the extension tube 220 or the bag attachment member 230. In the illustrated embodiment, the second sterile package 240B may be configured for separation from the extension tube 220. In some embodiments, the separable perimeter seam 241B may extend around the extension tube 220 or the bag attachment member 230 to form the seal 243B. As such, pulling the opposing tabs 242B/244B apart may separate the separable perimeter seam 241B from the extension tube 220. The second sterile package 240B may be formed of any suitable medical grade plastic sheet material and the separable perimeter seam 241B may be formed by a heat sealing process or any other suitable bonding process. As such, the seal 243B may also be formed by the heat sealing process or any other suitable bonding process.

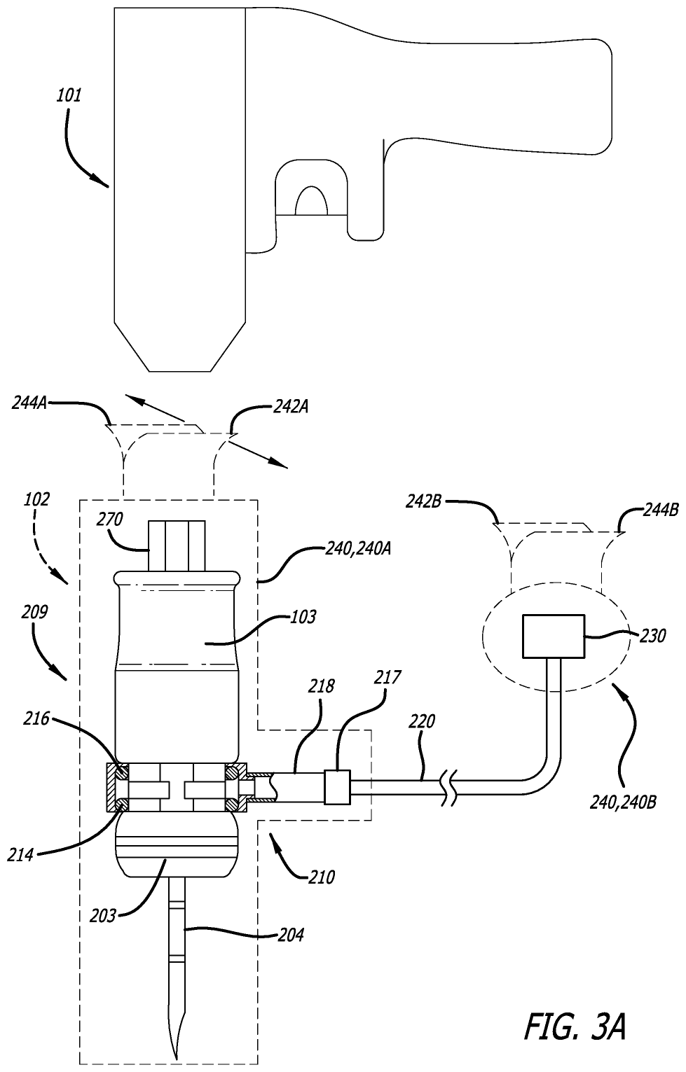

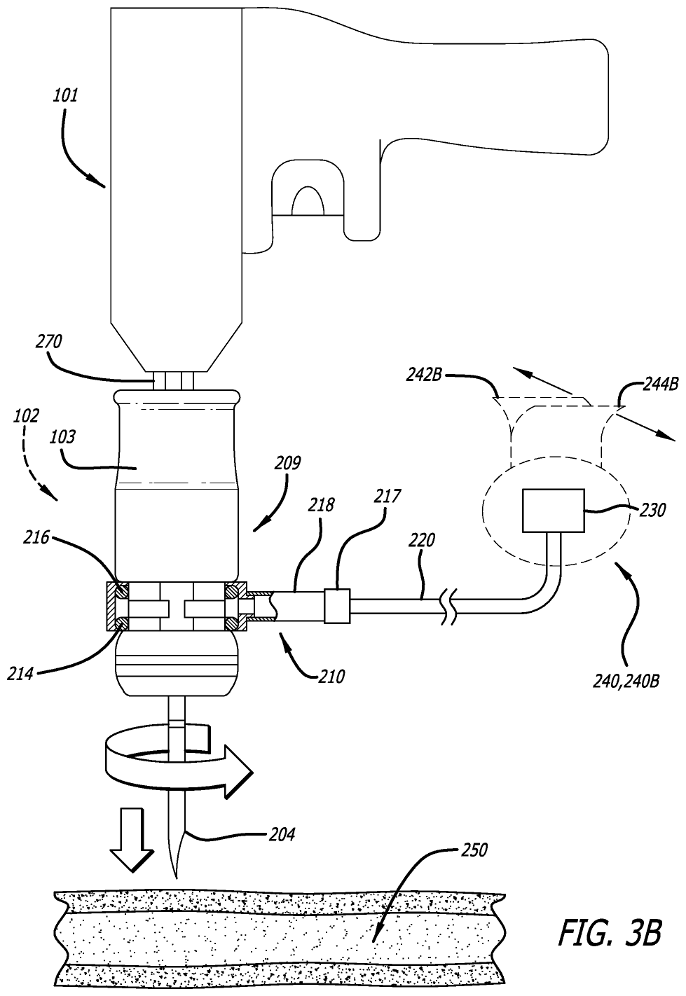

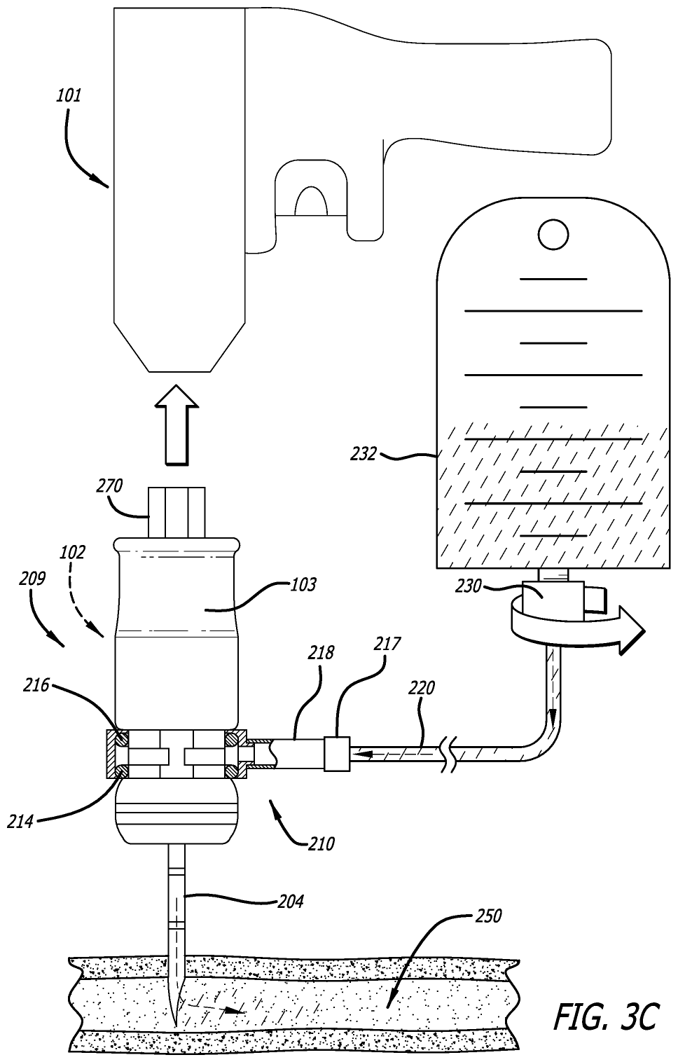

FIGS. 3A-3C illustrate a perspective view of accessing the intraosseous cavity 250 using the intraosseous access system 100 including the access assembly 209, in accordance with some embodiments. FIG. 3A illustrates the intraosseous access system 100 a pre-use state where the access assembly 209 including extension set 210 are disposed within the sterile package 240. In some embodiments, the access assembly 209 including extension set 210 may be detachably coupled to the driver 101. In some embodiments, the first sterile package 240A may be removed from the access assembly 209, and the intraosseous socket head 270 may be coupled to the driver 101. In some embodiments, the access assembly 209 including the extension set 210 includes a rotatable connection so as to allow the driver 101 to rotate the access assembly 209. In an embodiment, any one of the obturator assembly 102, the access assembly 209, the intraosseous socket head 270, the extension set 210, or the driver 101 may include a locking mechanism (not shown) to lock the obturator assembly 102 or the access assembly 209 to the driver 101 during use.

FIG. 3B illustrates the intraosseous access system 100 in an embodiment of a ready-to-use state, where the access assembly 209 is removed from the first sterile package 240A, the access assembly 209 is coupled with the driver 101, and where the bag attachment member 230 remains disposed within the second sterile package 240B. As illustrated in FIG. 3B, once the obturator assembly 102 and the access assembly 209, including the extension set 210, is coupled to the driver 101, the driver 101 may be actuated to rotate the access assembly 209, including the access needle 204, to access the intraosseous cavity 250. The first O-ring 216 and second O-ring 214 maintain the fluid-tight seal between the extension tubing hub 218 and access assembly 209 during rotation of the access assembly 209 while the extension tube hub 218 and extension tube 220 do not rotate.

FIG. 3C illustrates the intraosseous access system 100 in an embodiment of use. As illustrated, once the access assembly 209 has accessed the intraosseous cavity 250, the driver 101 may be decoupled from the access assembly 209. The bag attachment member 230 may be removed from the second sterile package 240B, and the fluid bag 232 may be coupled to the bag attachment member 230. In some embodiments, the locking mechanism (not shown) may be disengaged before the driver 101 is separated from the obturator assembly 102 and/or the access assembly 209. A volume of fluid may flow from the fluid bag 232 into the intraosseous cavity 250 through the extension set 210 under gravity flow. Advantageously, the intraosseous cavity 250 may be accessed, allowing fluid to flow into the intraosseous cavity 250 via fewer steps than may be typical, thereby reducing the potential of contamination and speeding up the process of delivering fluids to the intraosseous cavity 250.

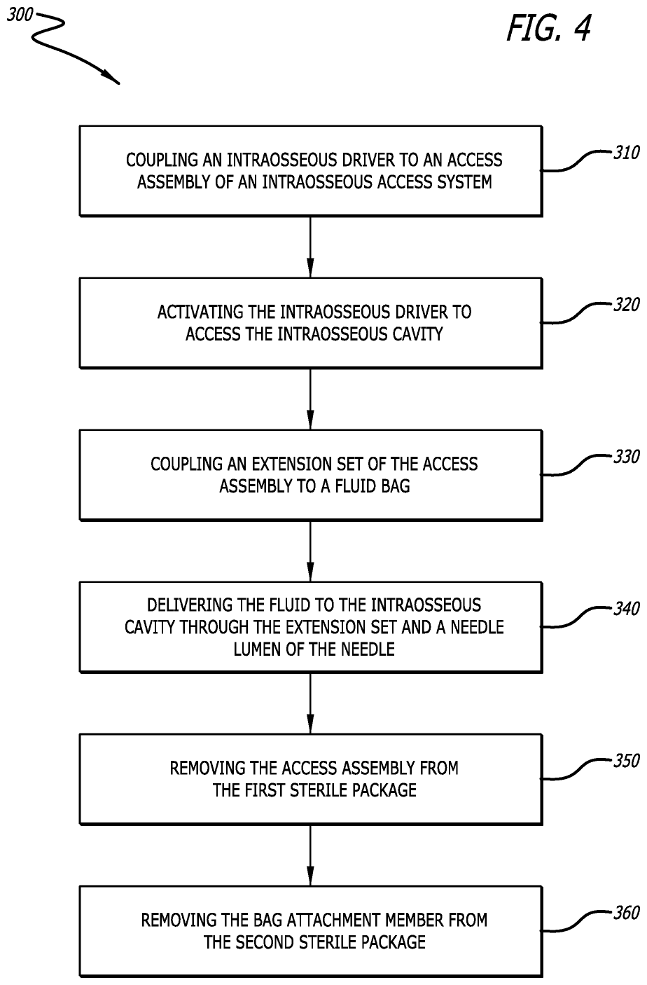

FIG. 4 illustrates a flow chart of an exemplary method 300 of accessing an intraosseous cavity that, according to some embodiments, may include all or any subset of the following steps, actions, or processes. The method 300 may include coupling an intraosseous driver to the access assembly of the intraosseous access system (block 310) so that activating the driver may causes the access assembly including the access needle to rotate.

The method 300 may further include activating the intraosseous driver to access the intraosseous cavity (block 320), where activating the intraosseous driver includes rotating the access needle of the access assembly. The method 300 may further include coupling the extension set of the access assembly to a fluid bag (block 330) where the fluid bag contains a fluid, and where the extension set is rotatably coupled with the access assembly to enable rotation of the access assembly independent of the extension set. The method 300 may further include delivering the fluid to the intraosseous cavity through the extension set and a needle lumen of the access needle (block 340). In some embodiments of the method 300, activating the intraosseous driver and delivering the fluid may occur simultaneously.

In some embodiments of the method 300, the intraosseous access system includes a first sterile package containing the access assembly, where at least a first portion of an extension tube of the extension set is contained within the first sterile package, and where at least a second portion of the extension tube is not contained within the first sterile package. In such embodiments, the method 300 may further include removing the access assembly from the first sterile package (block 350). In some embodiments of the method 300, the extension tube extends between a sterile interior of the first sterile package and a non-sterile exterior of the first sterile package, and the first sterile package forms a seal with the extension tube. In some embodiments of the method 300, removing the access assembly from the first sterile package may include breaching the seal between the first sterile package and the extension tube.

In some embodiments of the method 300, the intraosseous access system includes a second sterile package containing the bag attachment member of the extension set, where at least a third portion of the extension tube is contained within the second sterile package, and where at least a fourth portion of the extension tube is not contained within the second sterile package. In such embodiments, the method 300 may further include removing the bag attachment member from the second sterile package (block 360). In some embodiments of the method, the extension tube extends between a sterile interior of the second sterile package and a non-sterile exterior of the second sterile package, and the second sterile package forms a seal with the extension tube. In some embodiments of the method 300, removing the bag attachment member from the second sterile package may include breaching the seal between the second sterile package and the extension tube.

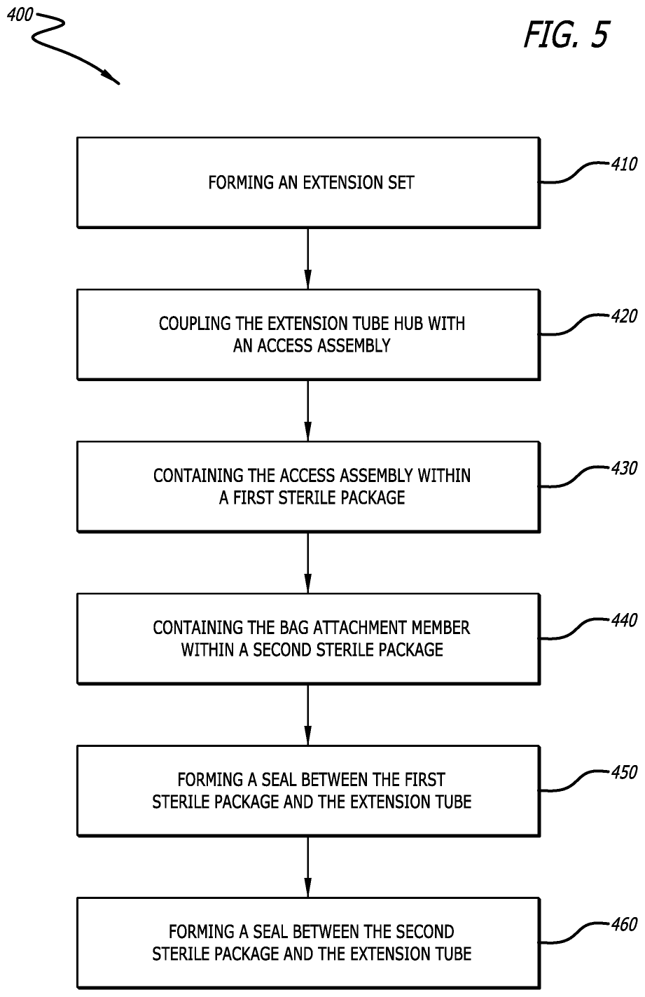

FIG. 5 illustrates a flow chart of an exemplary method 400 of manufacturing the intraosseous access system that, according to some embodiments, may include all or any subset of the following steps, actions, or processes. The method 400 may include forming an extension set (block 410), where extension set includes an extension tube having an extension tube hub at a first end and a bag attachment member at a second end. The method 400 may further include coupling the extension tube hub with an access assembly (block 420), where the access assembly includes an obturator assembly having an obturator and a needle assembly having an access needle. In some embodiments of the method 400, the obturator assembly is coupled with the needle assembly such that the obturator is disposed within a needle lumen of the access needle. According to some embodiments of the method 400, coupling the extension tube hub with the access assembly includes encapsulating the extension tube hub between the obturator assembly and the needle assembly and forming a rotating fluid-tight seal between the extension tube hub and the access assembly.

In some embodiments of the method 400, coupling the extension tube hub with the access assembly further includes defining a fluid pathway between the bag attachment member and the needle lumen.

The method 400 may further include containing the access assembly within a first sterile package (block 430). According to some embodiments of the method 400, at least a first portion of the extension tube is contained within the first sterile package and at least a second portion of the extension tube is not contained within the first sterile package.

The method 400 may further include containing the bag attachment member within a second sterile package (block 440) where the second sterile pack is separate from the first sterile package. According to some embodiments of the method 400, at least a third portion of the extension tube is contained within the second sterile package and at least a fourth portion of the extension tube is not contained within the second sterile package.

In some embodiments of the method 400 further includes forming a seal between the first sterile package and the extension tube (block 450) and in some embodiments of the method 400 further includes forming a seal between the second sterile package and the extension tube (block 460). In some embodiments, the method 400 may include obtaining the driver.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intraosseous access system, comprising:
an intraosseous driver;
an access assembly coupled with the driver, comprising:
an obturator assembly including an obturator; and
a needle assembly including an access needle, the needle assembly coupled with the obturator assembly such that the obturator is disposed within a needle lumen of the access needle; and
an extension set coupled with the access assembly, the extension set comprising extension tube extending between an extension tube hub at a proximal end and a bag attachment member at a distal end,
wherein the extension tube hub is coupled with the access assembly such that the bag attachment member is in fluid communication with the needle lumen.

2. The system according to claim 1, wherein the access assembly is rotatably coupled with the extension tube hub to enable the access assembly to rotate with respect to the extension set.

3. The system according to claim 1, wherein:
the needle assembly includes a fluid port in fluid communication with the needle lumen, and
the extension tube hub includes:
a first O-ring defining a first rotating seal with the needle assembly, the first O-ring disposed proximal the fluid port; and
a second O-ring defining a second rotating seal with the needle assembly, the second O-ring disposed distal the fluid port.

4. The system according to claim 1, wherein, during use, the bag attachment member is coupled with a fluid bag having a fluid therein.

5. The system according to claim 4, wherein, during use, the fluid flows from the fluid bag through the needle lumen.

6. The system according to claim 1, further comprising a first sterile package, wherein the access assembly is contained within the first sterile package.

7. The system according to claim 6, wherein the extension tube extends between a sterile interior of the first sterile package and a non-sterile exterior of the first sterile package.

8. The system according to claim 1, further comprising a second sterile package, wherein the bag attachment member is contained within the second sterile package.

9. The system according to claim 8, wherein the extension tube extends between a sterile interior of the second sterile package and a non-sterile exterior of the second sterile package.

10. A method of accessing an intraosseous cavity, comprising:
coupling an intraosseous driver to an access assembly of an intraosseous access system;
activating the intraosseous driver to access the intraosseous cavity, wherein activating the intraosseous driver includes rotating an access needle the access assembly;
coupling an extension set of the access assembly to a fluid bag containing a fluid, wherein the extension set is rotatably coupled with the access assembly to enable rotation of the access assembly with respect to the extension set; and
delivering the fluid to the intraosseous cavity through the extension set and a needle lumen of the needle.

11. The method according to claim 10, wherein activating the intraosseous driver and delivering the fluid occur simultaneously.

12. The method according to claim 10, wherein:
the intraosseous access system includes a first sterile package containing the access assembly,
at least a first portion of an extension tube of the extension set is contained within the first sterile package,
at least a second portion of the extension tube is not contained within the first sterile package, and
the method further comprises removing the access assembly from the first sterile package.

13. The method according to claim 12, wherein:
the extension tube extends between a sterile interior of the first sterile package and a non-sterile exterior of the first sterile package, and
the first sterile package forms a seal with the extension tube.

14. The method according to claim 10, wherein:
the intraosseous access system includes a second sterile package containing a bag attachment member of the extension set,
at least a third portion of the extension tube is contained within the second sterile package,
at least a fourth portion of the extension tube is not contained within the second sterile package, and
the method further comprises removing the bag attachment member from the second sterile package.

15. The method according to claim 14, wherein:
the extension tube extends between a sterile interior of the second sterile package and a non-sterile exterior of the second sterile package, and
the second sterile package forms a seal with the extension tube.

16. A method of manufacturing an intraosseous access system, comprising:
forming an extension set comprising an extension tube having an extension tube hub at a first end and a bag attachment member at a second end; and
coupling the extension tube hub with an access assembly, the access assembly including:
an obturator assembly having an obturator, and
a needle assembly having an access needle, wherein the obturator assembly is coupled with the needle assembly such that the obturator is disposed within a needle lumen of the access needle,
wherein coupling the extension tube hub with the access assembly includes:
encapsulating the extension tube hub between the obturator assembly and the needle assembly; and
forming a rotating fluid-tight seal between the extension tube hub and the access assembly.

17. The method according to claim 16, wherein coupling the extension tube hub with the access assembly further includes defining a fluid pathway between the bag attachment member and the needle lumen.

18. The method according to claim 16, further comprising containing the access assembly within a first sterile package such that:
at least a first portion of the extension tube is contained within the first sterile package,
at least a second portion of the extension tube is not contained within the first sterile package.

19. The method according to claim 18, wherein containing the access assembly within the first sterile package includes forming a seal between the first sterile package and the extension tube.

20. The method according to claim 16, further comprising containing the bag attachment member within a second sterile package, separate from the first sterile package, such that:
at least a third portion of the extension tube is contained within the second sterile package,
at least a fourth portion of the extension tube is not contained within the second sterile package.

21. The method according to claim 20, wherein containing the bag attachment member within the second sterile package includes forming a seal between the second sterile package and the extension tube.
